(19) United States
(12) Patent Application Publication
(10) Pub. No.: US 2008/0254520 A1
(43) Pub. Date: Oct. 16, 2008

(54) Method for reducing impurity level in mycophenolic acid fermentation

(76) Inventors: Eva Gulyas, Debrecen (HU); Gabor Balogh, Debrecen (HU); Janos Erdei, Debrecen (HU); Laszlo Toth, Balmazujvaros (HU); Boglarka Szikszai, Debrecen (HU); Alexandr Jegorov, Dobra Voda (CZ); Jiri Faustmann, Opava 6 (CZ)

Correspondence Address: KENYON & KENYON LLP, ONE BROADWAY, NEW YORK, NY, 10004, US

(21) Appl. No.: 12/082,484

(22) Filed: Apr. 11, 2008

Related U.S. Application Data
(60) Provisional application No. 60/923,079, filed on Apr. 11, 2007.
(60) Provisional application No. 60/998,341, filed on Oct. 9, 2007.
(60) Provisional application No. 61/019,036, filed on Jan. 4, 2008.

(51) Int. Cl. C12P 17/16; C07D 307/54; G01N 33/50; C07D 413/12; C12P 17/04

(52) U.S. Cl. 435/118; 549/305; 435/126; 436/93; 544/153

(57) ABSTRACT

The present invention relates to methods for reducing impurities of mycophenolic acid during fermentation by controlling the level of carbon source during fermentation of mycophenolic acid and for the isolation and use as a standard marker of the impurity homo-mycophenolic acid.

### CROSS-REFERENCE TO RELATED APPLICATIONS

This application claims the benefit of U.S. provisional application Ser. Nos. 60/923,079, filed Apr. 11, 2007, 60/998,341, filed Oct. 9, 2007 and 61/019,036, filed Jan. 4, 2008, hereby incorporated by reference.

### FIELD OF THE INVENTION

The present invention relates to methods for reducing impurities of mycophenolic acid during fermentation, especially MPA-IV (E-10-(4-hydroxy-6-methoxy-7-methyl-3-oxo-1,3-dihydro-isobenzofuran-5-yl)-4,8-dimethyl-dec-4,8-dienoic acid) and homo-MPA (E-8-(4-hydroxy-6-methoxy-7-methyl-3-oxo-1,3-dihydro-isobenzofuran-5-yl)-2,6-dimethyl-oct-6-enoic acid). The invention also relates to isolated homo-MPA and to its use as a reference marker and standard.

### BACKGROUND OF THE INVENTION

(E)-6-(4-hydroxy-6-methoxy-7-methyl-3-oxo- 1H-isobenzofuran-5-yl)-4-methyl-hex-4-enoic acid, Mycophenolic acid (“MPA”), of the following formula

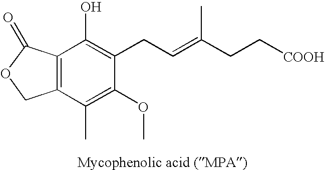

is an antibiotic produced by a biological process, i.e., fermentation done by a penicillium microorganism. There are several known species of penicillium, including P. brevicompactum, P. scabrum, P. nagemi, P. roqueforti, P. patris-mei and P. viridicatum (Clutterbuck, P. W., et al. 1932. “CLXXI. Studies in the biochemistry of microorganisms. XXIV. The metabolic products of the Penicillium brevi-compactum series,” Biochem.J. 26:1442-1458; and Frisvad, J. C. and Filtenborg, O. 1983. “Classification of terverticillate Penicillia based on profiles of mycotoxins and other secondary metabolites,” Appl.Environ.Microbiol. 46:1301-1310) in submerged and solid state fermentation).

During the fermentation process, certain starting materials are converted, following a series of biochemical reactions, into an end product, MPA.

Several types of fermentation processes are commonly used by those skilled in the art, for example, “batch fermentation”, “fed-batch fermentation”, or “continuous fermentation/chemostat”.

MPA is a starting material of mycophenolate mofetil (“MMF”), of the following formula

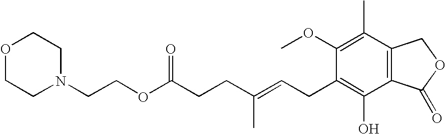

which is the 2-morpholinoethyl ester derivative of MPA that is approved for prophylaxis of rejection in patients receiving allogenic organ transplants.

GB patent no. 1157099, EP patent no. 1 624070 A1, and JP patent no. 59091891, disclose fermentation processes for preparation of MPA, using strain improvement or medium optimization for improvement of MPA productivity.

WO publication no. 2006/038218 discloses according to its abstract “the manufacture of MPA by fermentation under optimal fermentation parameters using a new strain of Penicillium arenicola.”

WO publication no. 2008/026883, which has a publication date that is after the present application's priority date, discloses according to its abstract “method for producing mycophenolic acid by culturing Pennicilium brevi-compactum in a culture solution comprising 3-9 g urea, carbon source, nitrogen source, and trace elements.”

J Chem. Soc. 2:365-73; 1982 reports an impurity of MPA of the following structure.

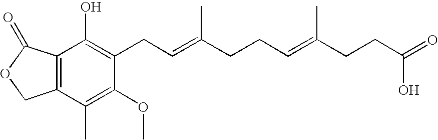

known as E-10-(4-hydroxy-6-methoxy-7-methyl-3-oxo-1,3-dihydro-isobenzofuran-5-yl)-4,8-dimethyl-dec-4,8-dienoic acid (“MPA-IV”), which is produced during the fermentation process for preparing MPA. This impurity shares structural similarities with MPA and can be esterified with morpholinoethanol when converting MPA to MMF, resulting in the homologue impurity of mycophenolate mofetil, having the following formula:

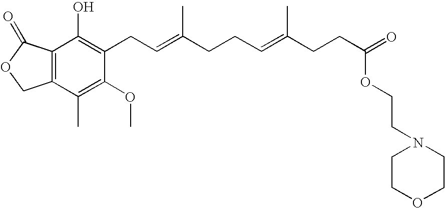

The product mixture of a chemical reaction is rarely a single compound with sufficient purity to comply with pharmaceutical standards. Therefore, the fermentation products in the broth can contain, in addition to MPA, additional compounds or impurities. These impurities may be, for example, intermediates of the reaction, such as MPA-IV, by-products of the reaction, products of side reactions, or degradation products. Impurities in MPA, or in any active pharmaceutical ingredient (“API”), such as MMF, are undesirable and, in extreme cases, might even be harmful to a patient being treated with a dosage form containing the API.

The purity of an API produced in a manufacturing process is critical for commercialization. The U.S. Food and Drug Administration (“FDA”) requires that process impurities be maintained below set limits. For example, in its ICH Q7A guidance for API manufacturers, the FDA specifies the quality of raw materials that may be used, as well as acceptable process conditions, such as temperature, pressure, time, and stoichiometric ratios, including purification steps, such as crystallization, distillation, and liquid-liquid extraction. See ICH Good Manufacturing Practice Guide for Active Pharmaceutical Ingredients, Q7A, Current Step 4 Version (Nov. 10, 2000).

At certain stages during processing of an API, such as MPA, it must be analyzed for purity, typically, by high performance liquid chromatography (“HPLC”) or thin-layer chromatography (“TLC”), to determine if it is suitable for continued processing and, ultimately, for use in a pharmaceutical product. The API need not be absolutely pure, as absolute purity is a theoretical ideal that is typically unatainable. Rather, the FDA requires that an API is as free of impurities as possible, so that it is as safe as possible for clinical use. For example, the FDA recommends that the amounts of some impurities be limited to less than 0.1 percent. See ICH Good Manufacturing Practice Guide for Active Pharmaceutical Ingredients, Q7A, Current Step 4 Version (Nov. 10, 2000).

Generally, side products, by-products, and adjunct reagents (collectively “impurities”) are identified spectroscopically and/or with another physical method, and then associated with a peak position, such as that in a chromatogram, or a spot on a TLC plate. See Strobel, H. A., et al., CHEMICAL INSTRUMENTATION: A SYSTEMATIC APPROACH, 953, 3d ed. (Wiley & Sons, New York 1989). Once a particular impurity has been associated with a peak position, the impurity can be identified in a sample by its relative position in the chromatogram, where the position in the chromatogram is measured in minutes between injection of the sample on the column and elution of the impurity through the detector. The relative position in the chromatogram is known as the “retention time.”

The retention time can vary about a mean value based upon the condition of the instrumentation, as well as many other factors. To mitigate the effects such variations have upon accurate identification of an impurity, practitioners often use “relative retention time” (“RRT”) to identify impurities. See supra Strobel at 922. The RRT of an impurity is calculated by dividing the retention time of the impurity by the retention time of a reference marker. The reference marker may be the API in which the impurity is present, or may be another compound that is either present in or added to the sample. A reference marker should be present in the sample in an amount that is sufficiently large to be detectable, but not in an amount large enough to saturate the column.

Those skilled in the art of drug manufacturing research and development understand that a relatively pure compound can be used as a “reference standard.” A reference standard is similar to a reference marker, except that it may be used not only to identify the impurity, but also to quantify the amount of the impurity present in the sample.

A reference standard is an “external standard,” when a solution of a known concentration of the reference standard and an unknown mixture are analyzed separately using the same technique. See supra Strobel at 924; Snyder, L. R., et al., INTRODUCTION TO MODERN LIQUID CHROMATOGRAPHY, 549, 2d ed. (John Wiley & Sons, New York 1979). The amount of the impurity in the sample can be determined by comparing the magnitude of the detector response for the reference standard to that for the impurity. See U.S. Pat. No. 6,333,198, hereby incorporated by reference.

The reference standard can also be used as an “internal standard,” i.e., one that is directly added to the sample in a predetermined amount. When the reference standard is an internal standard, a “response factor,” which compensates for differences in the sensitivity of the detector to the impurity and the reference standard, is used to quantify the amount of the impurity in the sample. See supra Strobel at 894. For this purpose, the reference standard is added directly to the mixture, and is known as an “internal standard.” See supra Strobel at 925; Snyder at 552.

The technique of “standard addition” can also be used to quantify the amount of the impurity. This technique is used where the sample contains an unknown detectable amount of the reference standard. In a “standard addition,” at least two samples are prepared by adding known and differing amounts of the internal standard. See supra Strobel at 391-393; Snyder at 571-572. The proportion of the detector response due to the reference standard present in the sample can be determined by plotting the detector response against the amount of the reference standard added to each of the samples, and extrapolating the plot to zero. See supra Strobel at 392, Figure 11.4. The response of a detector in HPLC (e.g., UV detectors or refractive index detectors) can be and typically is different for each compound eluting from the HPLC column. Response factors, as known, account for this difference in the response signal of the detector to different compounds eluting from the column.

As is known by those skilled in the art, the management of process impurities is greatly enhanced by understanding their chemical structures and synthetic pathways, and by identifying the parameters that influence the amount of impurities in the final product.

Therefore, it would be beneficial to develop a method that reduces the level of impurities in MPA during fermentation. Also, it would be beneficial to isolate, identify and quantify these impurities.

### SUMMARY OF THE INVENTION

In one embodiment, the present invention encompasses a process for reducing impurity formation during fermentation of mycophenolic acid (MPA) comprising: controlling the level of carbon source during fermentation of MPA, wherein the carbon source is maintained at an amount of about 0.8% to about 0.02% w/w.

In another embodiment, the present invention encompasses a process for preparing mycophenolate mofetil (MMF) comprising preparing MPA according to the process of the present invention, and converting it to MMF.

In another embodiment, the present invention encompasses isolated E-8-(4-hydroxy-6-methoxy-7-methyl-3-oxo-1,3-dihydro-isobenzofuran-5-yl)-2,6-dimethyl-oct-6-enoic acid (“Homo-MPA”) of the following formula:

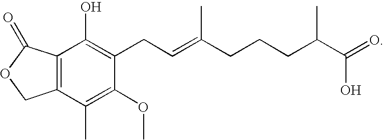

In yet another embodiment, the present invention encompasses a process of determining the presence of Homo-MPA in a sample of MPA by a process comprising carrying out HPLC or TLC with the Homo-MPA as a reference marker.

In another embodiment, the present invention encompasses a process of determining the amount of Homo-MPA in a sample of MPA comprising Homo-MPA and MPA by a process comprising carrying out HPLC with Homo-MPA as a reference standard.

In another embodiment, the present invention encompasses a method for preparing mycophenolic acid comprising: preparing a fermentation broth containing a mycophenolic acid producing micro-organism; fermenting of mycophenolic acid while feeding nutrients to the fermentation broth; controlling the level of carbon source in the fermentation broth at an amount of about 0.02% to about 0.8% w/w during the fermentation stage of mycophenolic acid; harvesting and recovering the mycophenolic acid from the fermentation broth.

In yet another embodiment the present invention encompasses an isolated compound of following structure:

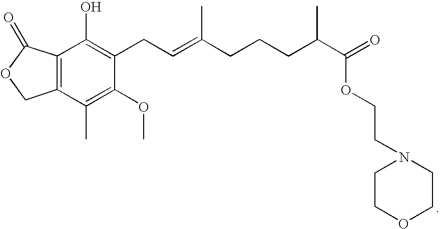

### BRIEF DESCRIPTION OF THE FIGURES

FIG. 1 displays the time course of MPA production, MPA-IV and homo-mycophenolic acid level and the pH during batch fermentation process by Penicillium sp. strain (accession number CCM 8364).

FIG. 2. displays a batch fermentation of mycophenolic acid by Penicillium sp. strain (accession number CCM 8364), according to GB 1,157,099.

FIG. 3. displays a batch fermentation of mycophenolic acid by Penicillium brevicompactum.

FIG. 4 displays the time course of MPA production, MPA-IV and homo-mycophenolic acid level and the pH during fed-batch fermentation process by Penicillium sp. strain (accession number CCM 8364).

FIG. 5 displays the time course of MPA production, MPA-IV and homo-mycophenolic acid level and the pH during an improved fed-batch fermentation process by Penicillium sp. strain (accession number CCM 8364).

FIG. 6 displays a table of glucose level vs. time for the improved fed-batch fermentation process by Penicillium sp. strain (accession number CCM 8364).

FIG. 7 displays a graph of the time course of glucose and ammonia levels for the improved fed-batch fermentation process by Penicillium sp. strain (accession number CCM 8364).

FIG. 8 displays an improved fed-batch fermentation of Penicillium brevicompactum.

FIG. 9 displays a ¹H NMR spectrum of homo-mycophenolic acid.

FIG. 10 displays a ¹³C NMR spectrum of homo-mycophenolic acid.

### DETAILED DESCRIPTION OF THE INVENTION

Typically, the fermentation of mycophenolic acid (MPA) includes the following stages: 1) a Growth phase, 2) a Production phase and 3) harvesting.

The present invention relates to a fed-batch fermentation process, where during the production phase a feeding process is conducted, thus a feeding period exists as a part of the production phase.

During the feeding period the level of the carbon source is controlled/maintained, thus leading to a substantially low level of side-products or impurities, specifically, E-10-(4-hydroxy-6-methoxy-7-methyl-3-oxo-1,3-dihydro-isobenzofuran-5-yl)-4,8-dimethyl-dec-4,8-dienoic acid (“MPA-IV”) and/or E-8-(4-hydroxy-6-methoxy-7-methyl-3-oxo-1,3-dihydro-isobenzofuran-5-yl)-2,6-dimethyl-oct-6-enoic acid (“homo-MPA”). Hence, the method of the present invention can be considered as a method that reduces the amount of these impurities as compared to their amount produced in fermentation processes where the level of the carbon source isn't maintained at all (examples 1-3) or maintained but not at such low level.

The level of impurities, including by-products produced in the fermentation stage, has major impact on the overall economy of the production process by influencing the efficiency of subsequent purification steps. In addition, elimination of these impurities from the fermentation broth often requires sophisticated purification methods, due to their structure similarity. These purification methods are also expensive to perform and provide low yields during downstream processing. Thus, developing a method for reducing impurities during fermentation of mycophenolic acid can provide increasing economical benefits.

In addition, since mycophenolic acid and mycophenolate mofetil are high dose medicines, the level of impurities allowed in MPA/MMF will be more strictly controlled. See attachment 1 of ICH Q3A(R) guideline issued on February 2002.

The method of reducing the level of MPA-IV and homo-mycophenolic acid in the preparation of mycophenolic acid as in the current invention comprises controlling the level of the carbon source during fermentation of MPA, wherein the carbon source is maintained at an amount of about 0.02% to about 0.8% w/w, preferably at about 0.05% to about 0.5% w/w.

As used herein the term “w/w” represents a way to express the concentration of the carbon source (in units of gram/gram of fermentation broth) in the aqueous fermentation broth as measured a the chemical analyzer equipment (e.g. a BioProfile® 100 chemical analyzer from Nova Biomedical, USA).

Preferably, the level of the carbon source is controlled/maintained during the feeding period of the fermentation process. Before this period, the carbon source may be about 1% to about 5% w/w, more preferably about 2% to about 3% w/w.

Preferably, the carbon source is carbohydrate or a combination of carbohydrates. More preferably, the carbon source is starch or molasses, even more preferably, glucose, sucrose, maltose, glycerol. Most preferably, the carbon source is glucose.

Maintaining this level of carbon source is typically done by sampling the fermentation broth to measure the level of the carbon source in order to determine if to continue feeding. The sample taken from the fermentation broth can be centrifuged at 3000 rpm for 10 seconds, filtered and the amount of the carbon source is measured using a BioProfile® 100Plus biochemical analyzer (through amperometric enzyme electrode). Alternatively, the carbon source level can be monitored on-line using, for example, infra-red or near infra-red spectroscopy.

The process of the invention results in reduced levels of MPA-IV and homo-MPA in the fermentation broth as well as in the final MPA product. Preferably, the amount of MPA-IV and homo-MPA in the obtained fermentation broth is less than about 0.5% area by HPLC, more preferably, less than about 0.3% area by HPLC, even more preferably, less than about 0.2% area by HPLC, and most preferably, less than about 0.15% area by HPLC. The amount of these impurities is calculated by dividing the area under the peak of each one of the impurities by the area under the peak of MPA. Thus, the amount of each impurity is expressed as area % relative to the amount of MPA.

Preferably the fermentation broth comprising MPA contains from about 0.001% to about 0.5%, preferably about 0.01 to about 0.5%, more preferably about 0.05 to about 0.2%, and most preferably about 0.05 to about 0.15% area by HPLC of MPA-IV and/or homo-MPA.

Further reduction of the impurities level, specifically of MPA-IV and homo-MPA may be achieved by stopping the feeding during the final 20 to 96 hours of the production stage of mycophenolic acid (i.e. before harvesting). Preferably, the feeding period ends about 24 to 96 hours, more preferably at about 24 hours to about 48 hours prior to harvesting.

This operation further improves the purity of the obtained mycophenolic acid in the fermentation broth, specifically from homo-MPA and MPA-IV, compared to the purity obtained when the feeding period is stopped just before harvesting, as exemplified in examples 5 and 6 vs. examples 1-3.

Another benefit for controlling the carbon source level is to increase MPA production rate—lower levels of carbon source result in higher production rates of MPA, as exemplified when comparing the final productivity rate of mycophenolic acid in example 1 with examples 4-5 for Penicillium sp. (MPA production rate is 30 μg/g/h relative to 33-39 μg/g/h, respectively) and example 3 with example 6 for Penicillium brevicompactum (MPA production rate is 6.4 μg/g/h compared to 7.8 μg/g/h, respectively).

As used herein, the term “μg/g/h” refers to the “rate” of MPA production: μg MPA produced per 1 g fermentation broth in one hour. The MPA production rate may be calculated as follows: final titer (μg/g) divided by the fermentation time (hours).

Another benefit for maintaining a low level of the carbon source is to maximize the yield of the product. The yield is expressed as the final titer measured by HPLC.

In a preferred embodiment of the present invention, the fermentation medium contains a starting level of a carbon source in the growth period, which is about 1% to about 5% w/w of the fermentation medium (the fermentation medium includes the whole broth including liquids, solids, microbial cells). This level is then reduced by the microorganisms, and is maintained at about 0.02% to about 0.8% w/w during the production period, preferably at about 0.05% to about 0.5% w/w.

The fermentation medium contains also a source of nitrogen. Preferably, the nitrogen source is also fed batch-wise.

The fermentation medium can contain additional nutrients to help improve the productivity. These other nutrients include mineral salts, microbial growth factors, a source of phosphorous and a buffer.

Sources of mineral salts (e.g. ammonium, calcium, iron, zinc, copper, magnesium, manganese, sodium or potassium salts) include magnesium sulfate, manganese dichloride, ferrous sulfate, zinc chloride, copper II sulfate, ammonium sulfate, potassium dihydrogen phosphate, sodium chloride and calcium carbonate. A combination of these sources can be used. Preferably the source is magnesium sulfate

Examples of organic or inorganic nitrogen include nitrate, urea, ammonium salts, amino acids, vegetable flours and corn steep liquor. Specific examples of these nitrogen sources are further disclosed in WO2008/026883, incorporated herein by reference. Preferably, glycine and corn steep liquor are used.

Examples of microbial growth factors include yeast extract and vitamins. Examples of organic or inorganic phosphorous include potassium dihydrogen phosphate, sodium dihydrogen phosphate, potassium hydrogen phosphate and sodium hydrogen phosphate.

In one embodiment a combination of at least one nitrogen source, at least one phosphorous source, at least one mineral salt and at least one carbon source is used. Preferably a combination of glycine, corn-steep liquor, potassium dihydrogen phosphate, methionine, magnesium sulphate, and potato starch is used.

More preferably, the fermentation medium contains about 1-5% glucose, 0.1% to about 3% w/w glycine, about 0.1% to about 3% w/w corn-steep liquor, about 0.01% to about 0.5% w/w potassium dihydrogen phosphate, about 0.1% to about 1% w/w methionine and about 0.1% to about 1% w/w magnesium sulphate.

Preferably, the above fermentation medium is inoculated with about 5% to about 20% by weight of vegetative culture of mycophenolic acid producer strain. Preferably, the strain is a Penicillium sp. fungal strain of one of the following species: P. brevi-compactum, P. scabrum, P. nagemi, P. roqueforti, P. patris-mei and P. viridicatum or a derivative thereof, more preferably the Penicillium sp. strain (accession number CCM 8364-Czech Collection of Microorganisms (CCM) at Masaryk University, Brno, Czech Republic) or a derivative thereof.

The culture is then preferably mixed, aerated and the temperature is maintained at about 21° C. to about 29° C.

The obtained MPA is then isolated from the fermentation broth after the harvesting. Isolation can be done, for example, according to the process disclosed herein, by obtaining an alkaline fermentation broth by raising the pH at the end of fermentation and filtering the obtained broth. Subsequently, acidifying the obtained liquid and filtering the now acidic liquid, and re-suspending the filtrate in water and adjusting the pH to an alkaline pH.

The isolated MPA can then be converted to MMF. The conversion can be done, for example according to the process described in WO 2005/105771.

Preferably, the amount of MPA-IV and homo-MPA in the obtained MPA is less than about 0.5% area by HPLC, more preferably, less than about 0.3% area by HPLC, even more preferably, less than about 0.2% area by HPLC, and most preferably, less than about 0.15% area by HPLC. The amount of these impurities is calculated by dividing the area under the peak of each one of impurities by the area under the peak of MPA. Thus, the amount of each impurity is expressed as area % relative to the amount of MPA.

In another embodiment, the present invention encompasses isolated E-8-(4-hydroxy-6-methoxy-7-methyl-3-oxo-1,3-dihydro-isobenzofuran-5-yl)-2,6-dimethyl-oct-6-enoic acid (“homo-MPA”) of the following formula:

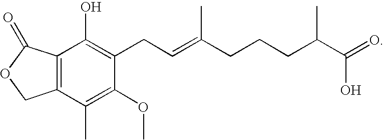

Preferably, the isolated homo-MPA is solid, more preferably, it is crystalline.

As used herein, the term “isolated” in reference to homo-MPA corresponds to homo-MPA that is physically separated from the fermentation broth. For example, the separation can be done by extractions and filtrations.

Preferably, the isolated homo-MPA of the present invention is separated from MPA thereby providing a composition of homo-MPA containing less than about 5%, preferably less than about 2%, and even more preferably less than about 1%, by weight, of MPA. Preferably, the homo-MPA composition comprises about 5% to about 0.05%, more preferably, about 2% to about 0.1%, most preferably, about 1% to about 0.1% by weight of MPA.

The content of MPA in homo-MPA is measured by HPLC.

The isolated homo-MPA of the present invention can be characterized by data selected from a group consisting of: ¹H NMR (400 MHz, CDCl₃) δ (ppm): 1.15, 1.35, 1.42, 1.63, 1.77, 1.98, 2.15, 2.43, 3.39, 3.77, 5.19, 5.20, and 7.68; ¹³C NMR (100 MHz, CDCl₃) δ (ppm): 11.5, 16.0, 16.8, 22.6, 25.2, 33.0, 39.1, 39.4, 61.0, 70.0106.4, 116.7, 122.2, 122.5, 135.5143.9, 153.7, 163.7, 172.9, and 182.3, and combination thereof.

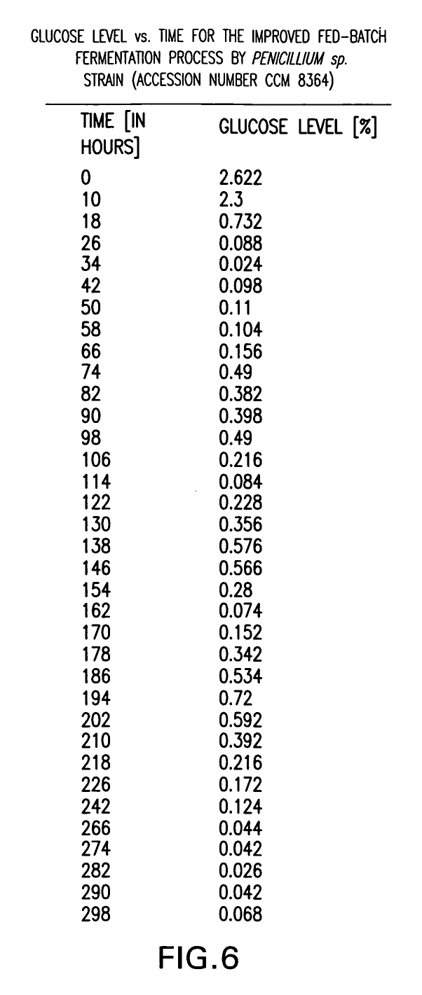

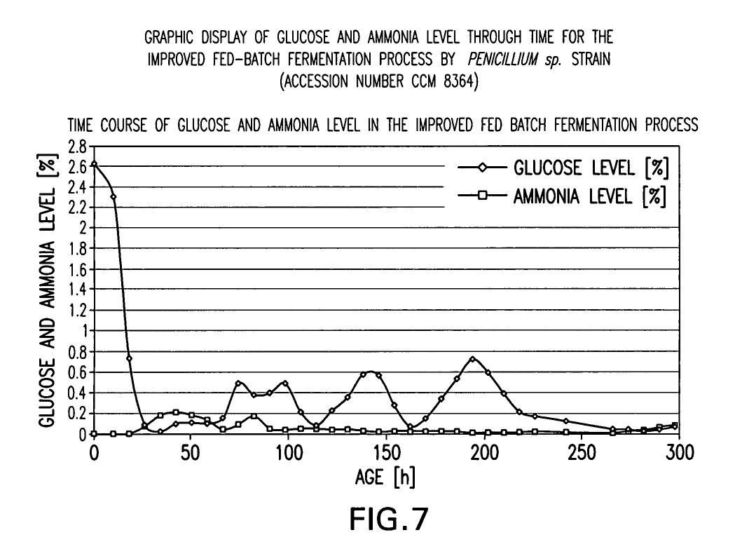

The isolated homo-MPA of the present invention may also be characterized by an ¹H NMR spectrum as depicted in FIG. 6. The isolated homo-MPA of the present invention may be further characterized by a ¹³C NMR spectrum as depicted in FIG. 7.

Homo-MPA is a side-product formed during the fermentation process for preparing MPA. It can be isolated from the fermentation broth, for example, by a process comprising a) providing a concentrate from of the fermentation broth comprising mycophenolic acid and homo-MPA, b) purifying the concentrate by column chromatography, and c) recovering the purified homo-mycophenolic acid.

Preferably, the purification by column chromatography is done by eluting the homo-MPA from a column comprising a resin, for example silica-gel, modified silica-gel, absorbent resin or ion exchange resin. More preferably, the resin is silica gel.

The purification is done by using a mixture of organic solvents such as, mixtures of polar and a-polar solvents, preferably the polar solvent is methanol or ethanol and the a-polar solvent is ether or dichloromethane. More preferably the eluent is a mixture of dichloro-methane and methanol. Most preferably, the eluent is a gradient eluent, wherein the amount of methanol is increased from 0 to about 5% v/v.

The eluted fractions are then tested for the presence of homo-MPA by TLC (benzene:acetic acid, 9:1 v/v), followed by selecting the fractions that contain homo-MPA. The recovery of homo-MPA from the selected fractions is done by evaporating the solvent.

Preferably, the recovered purified homo-MPA can be further purified by reversed-phase chromatography, more preferably by preparative HPLC, followed by crystallization from a solvent selected from a group consisting of: a mixture of methanol and water, a mixture of acetonitrile and water, a mixture of THF and water. More preferably, the solvent is a mixture of methanol and water or of acetonitrile and water.

The isolated homo-MPA can then be used to test the purity of MPA by using it as a reference marker and standard.

In one embodiment, the present invention encompasses a process of determining the presence of homo-MPA in a sample of MPA by a process comprising carrying out HPLC or TLC with the homo-MPA as a reference marker.

Preferably, the process comprises (a) measuring by HPLC or TLC the relative retention time or factor (referred to as RRT, or RRF, respectively) corresponding to the homo-MPA in a reference marker sample; (b) determining by HPLC or TLC the relative retention time corresponding to homo-MPA in a sample comprising homo-MPA and MPA; and (c) identifying homo-MPA in the sample by comparing the relative retention time or factor (RRT or RRF) of homo-MPA as measured in step of step (a) to the RRT or RRF of step (b).

In another embodiment, the present invention encompasses a process of determining the amount of homo-MPA in a sample of MPA comprising homo-MPA and MPA by a process comprising carrying out HPLC with homo-MPA as a reference standard.

Preferably, the above process comprises: (a) measuring by HPLC the area under a peak corresponding to the homo-MPA in a reference standard comprising a known amount of homo-MPA; (b) measuring by HPLC the area under a peak corresponding to homo-MPA in a sample comprising homo-MPA and MPA; and (c) determining the amount of homo-MPA in the sample by comparing the area of step (a) to the area of step (b).

As seen from its structure, homo-MPA is a close homologue of mycophenolic acid. Hence, since both acids (MPA and homo-MPA) have terminal carboxy groups, they can both be esterified with morfolino ethanol, when converting MPA to MMF. The respective morpholino ethanol ester of homo-MPA of the following formula:

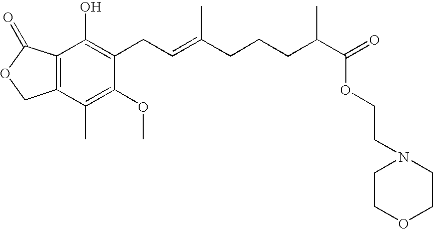

is thus a potential impurity in the MMF API.

Accordingly, the preparation of MMF having a reduced level of the ester of homo-MPA can be done after testing the purity of MPA, by selecting a batch of MPA that contains low levels of homo-MPA.

Having described the invention with reference to certain preferred embodiments, other embodiments will become apparent to one skilled in the art from consideration of the specification. The invention is further defined by reference to the following examples describing in detail the method for reducing impurity level in mycophenolic acid fermentation. It will be apparent to those skilled in the art that many modifications, both to materials and methods, may be practiced without departing from the scope of the invention.

### EXAMPLES

HPLC Method
Column: Reversed phase silica gel C8 (250*4.6 mm, 5 um)
Eluent A: 0.005 M phosphoric acid, pH 2.5
Eluent B: acetonitrile
Gradient elution, t(min)/B %: 0/30, 5/30, 10/37, 13/40, 23/60, 30/60, 35/100, 35.5/100, 45/30
Flow rate: 1.5 ml/min
Column temperature: 45 ° C.
Sample organizer: 10 ° C.
Detector: wavelength at 250 nm
Injection volume: 10 μl
Duration: 45 min
Diluent: acetonitrile:water (9:1) for fermentation broth

Retention times:

| | |
|---|---|
| MPA | 11.8 min |
| Homo MPA | 21.7 min |

Detection limit: 0.004%
Quantification limit:0.01%
Sample preparation: Fermentation broth was extracted with acetonitrile:water (9:1) for 15 minutes in an ultrasonic bath, and filtered on 0.45 um porosity filter.
Concentration is at about 400 μg/ml.
Relative response factors to MPA (at 250 nm):

| | |
|---|---|
| Homo MPA | 0.865 |
| Other impurities | 1.00. |

NMR spectroscopy
Instrument: Varian^{UNITY} Inova-400 at 399.87 MHz for ¹H, and at 100.55 MHz for ¹³C, (CDC13, 30° C.).

### Example 1

### Batch Fermentation of Mycophenolic Acid

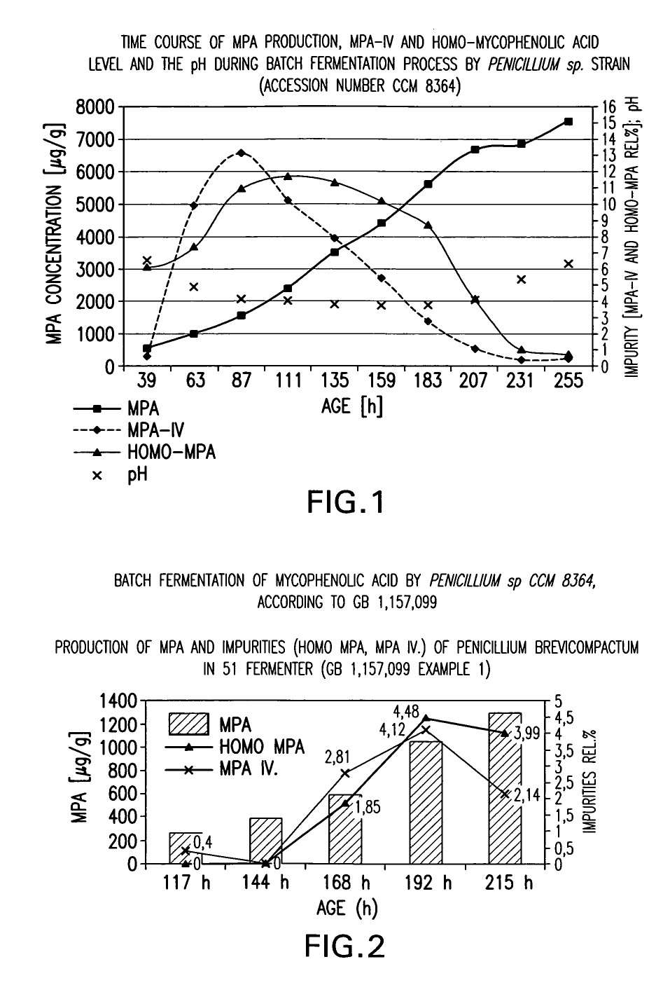

Medium containing 8% w/w potato starch, 12% w/w maltose, 1.5% w/w glycine, 0.3% w/w potassium dihydrogen-phosphate, 0.05% w/w methionine and 0.1% w/w magnesium-sulphate was inoculated by 10% vegetative culture of mycophenolic acid producer penicillium strain (Penicillium sp. having accession number CCM 8364). The culture was mixed and aerated and the temperature was maintained at 25 ±2° C. for 10 days. From the second day, mycophenolic acid concentration, MPA-IV and homo-mycophenolic acid level and the pH was measured (by HPLC and potentiometric electrode respectively). As a result of excess of nutrients in the batch medium, the MPA-IV and homo-mycophenolic acid level raised over 10% and was above 1 relative % (referred to the MPA level) up to the 8^{th} day, and then remained about 0.5% at the end of the fermentation. The final productivity (titer measured by HPLC), was 7.5 g/l of MPA and the average MPA production rate was 30 μg/g/h. At the end of the fermentation the pH was raised to above 6.0. See FIG. 1.

### Example 2

### Comparative Example: Batch Fermentation of Mycophenolic Acid, According to GB 1,157,099

5 litres of medium containing: 100 g/l glucose, 3.72 g/l ammonium-nitrate, 5.0 g/l potassium-hydrogen-phosphate, 1 g/l magnesium-sulphate and 2.0 ml/l trace element concentrate were inoculated with spores of the organism Penicillum sp. (strain No. CCM 8364) in a fermenter. The culture was maintained at a temperature of 25° C., with a stirring rate of 712 r.p.m. and with an air flow of ½ volume/volume/minute. After 93 hours, 65 ml of 80% (w/v) glucose solution was added. The concentration of mycophenolic acid, MPA-IV and homo-mycophenolic acid were measured during 117-215 hours. The level of MPA-IV and homo-mycophenolic acid raised to above 4% after 8 days then dropped to about 4% and 2%, respectively, after 215 hours (9 days). The final MPA titer was 1.3 g/l (see FIG. 2).

### Example 3

### Batch Fermentation of Mycophenolic Acid

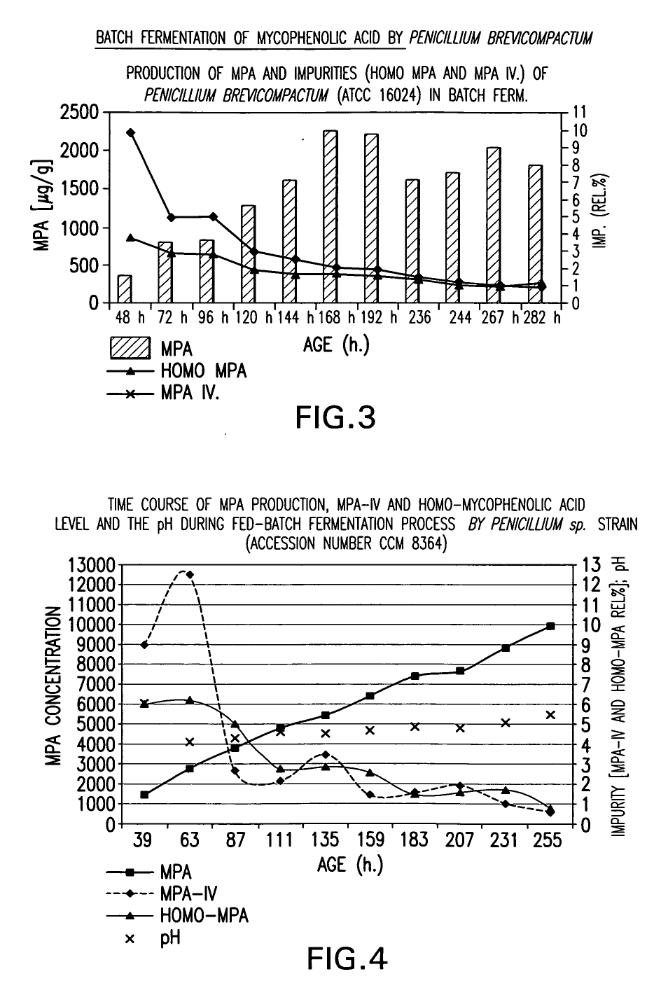

4.5 litres of medium containing 8% w/w potato starch, 12% w/w maltose, 1.5% w/w glycine, 0.3% w/w potassium dihydrogen-phosphate, 0.05% w/w methionine and 0.1% w/w magnesium-sulphate was inoculated by 10% vegetative culture of the organism Penicillum brevicompactum ATCC 16024. The culture was mixed with a stirring rate of 700-1000 r.p.m. and aerated with an air flow of 0.4-0.7 volume/volume/minute and the temperature was maintained at 25 ±2° C. for 12 days. From the second day, mycophenolic acid concentration, MPA-IV and homo-mycophenolic acid level were measured by HPLC. The MPA-IV and homo-mycophenolic acid level declined from about 10% and 3% area % relative to the main peak (of MPA) measured by HPLC, respectively, and reached about 1 relative % (referred to the MPA level) at the 9^{th} day, and then remained about the same until the end of the fermentation. The final productivity (titer measured by HPLC), was 1.8 g/l of MPA and the average MPA production rate was 6.4 μg/g/h (see FIG. 3).

### Example 4

### Fed-Batch Fermentation of Mycophenolic Acid (the Level of Carbon Source was Controlled and Feeding was Stopped at the End of the Fermentation)

Medium containing 3% w/w glucose, 1.5% w/w glycine, 0.5% w/w corn-steep liquor, 0.2% w/w potassium dihydrogen-phosphate, 0.1% w/w methionine and 0.1% w/w magnesium-sulphate was inoculated by 10% vegetative culture of mycophenolic acid producer penicillium strain (Penicillium sp. having accession number CCM 8364). The culture was mixed and aerated and the temperature was maintained at 25 ±2° C. for 10 days. The glucose level of the fermentation was maintained in the range of 0.02-0.8% w/w by feeding of the carbon source. Batch-wise feeding of CSL (0.05-0.2%/day from the 4th day) and glycine (0.1-0.3%/day from the 4th day) was also applied. From the second day, mycophenolic acid concentration, MPA-IV and homo-mycophenolic acid level and the pH was measured (by HPLC and potentiometric electrode, respectively). As a result of carbon source level controlled by the feeding, the MPA-IV and homo-mycophenolic acid levels continuously decrease till the end of the fermentation and reached about 0.5% area by HPLC at final level. The final productivity/titer was 9.9 g/l of MPA and the average MPA production rate was 39 μg/g/h. At the end of the fermentation the pH has not increased significantly (See FIG. 4)

### Example 5

### Improved Fed-Batch Fermentation of Mycophenolic Acid (the Level of Carbon Source was Controlled and Feeding was Stopped at 24-48 h before Harvest)

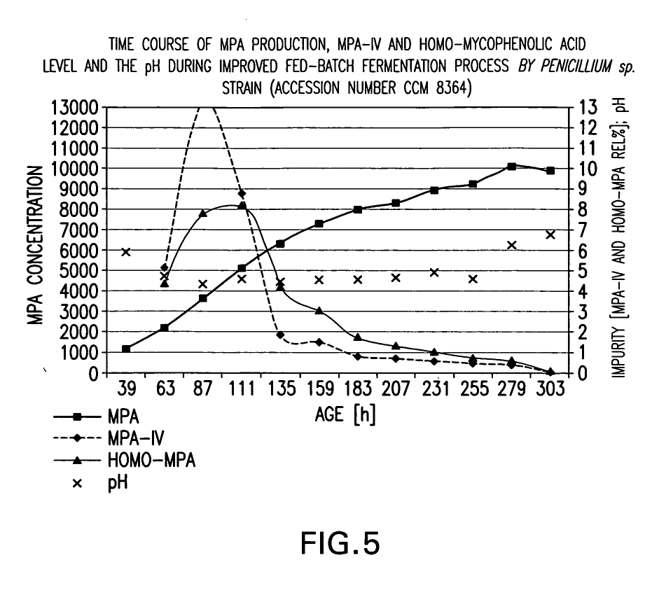

Medium containing 3% w/w glucose, 1.5% w/w glycine, 0.5% w/w corn-steep liquor, 0.2% w/w potassium dihydrogen-phosphate, 0.1% w/w methionine and 0.1% w/w magnesium-sulphate was inoculated by 10% vegetative culture of mycophenolic acid producer penicillium strain (Penicillium sp. having accession number CCM 8364). The culture was mixed and aerated and the temperature was maintained at 25 ±2° C. for 13 days. The glucose level of the fermentation was maintained in the range of 0.02-0.8% w/w by feeding of the carbon source. Batch-wise feeding of CSL (0.05-0.2%/day from the 4th day) and glycine (0.1-0.3%/day from the 4^{th} day) was also applied. Feeding of the nutrients was stopped on the 10^{th} day. From the second day, mycophenolic acid concentration, MPA-IV and homo-mycophenolic acid level and the pH were measured (by HPLC and potentiometric electrode, respectively). As a result of the controlled level of the carbon source by the feeding and ceasing of the supply during the last 24-48 hours of the fermentation, the MPA-IV and homo-mycophenolic acid levels continuously decrease till the end of the fermentation and reached a final level of less than about 0.15% area by HPLC. The final productivity was 10.0 g/l of MPA and the average MPA production rate was 33 ug/g/h. At the end of the fermentation the pH was raised to above 6.5. (See FIG. 5).

### Example 6

### Improved Fed-Batch Fermentation of Mycophenolic Acid (the Level of Carbon Source was Controlled and Feeding was Stopped at 24-48 h before Harvest)

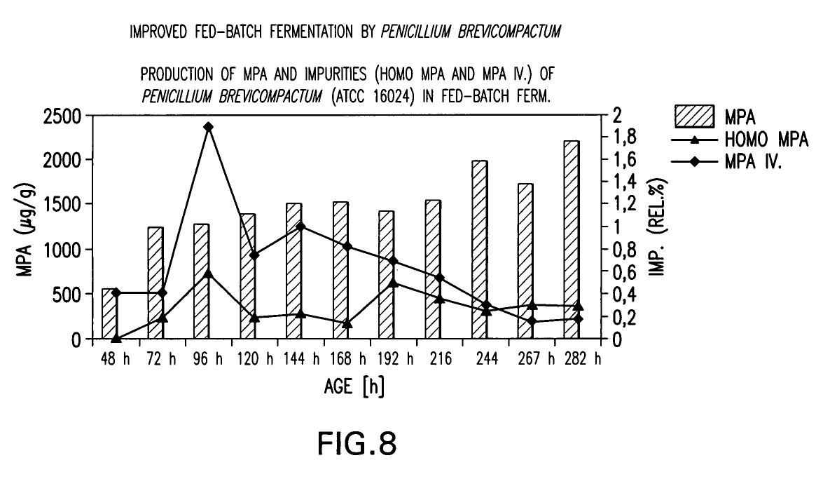

4.5 litres of medium containing 3% w/w glucose, 2.0% w/w glycine, 0.5% w/w corn-steep liquor (CSL), 0.2% w/w potassium dihydrogen-phosphate, 0.05% w/w methionine and 0.1% w/w magnesium-sulphate was inoculated by 10% vegetative culture of the organism Penicillum brevicompactum ATCC 16024. The culture was mixed with a stirring rate of 700-1000 r.p.m. and aerated with an air flow of 0.4-0.7 volume/volume/minute and the temperature was maintained at 25 ±2° C. for 12 days. The glucose level of the fermentation was maintained in the range of 0.02-0.8% w/w by feeding of glucose. Feeding of the glucose was stopped on the 10^{th} day. Batch-wise feeding of CSL (0.1-0.15-% /day from the 4^{th} day till the 9^{th} day) and glycine (0.1-0.2%/day from the 4^{th} day till the 9^{th} day) was also applied. From the second day, mycophenolic acid concentration, MPA-IV and homo-mycophenolic acid level was measured by HPLC. Feeding of the nutrients was stopped on the 10^{th} day. As a result of the controlled level of the carbon source by the feeding and ceasing of the supply during the last 48 hours of the fermentation, the MPA-IV and homo-mycophenolic acid levels increased and then continuously decrease till the end of the fermentation and reached a final level of less than about 0.3% area by HPLC. The final productivity was 2.2 g/l of MPA and the average MPA production rate was 7.8 ug/g/h.(see FIG. 8).

### Example 7

### Summary of Results

During fermentation, the impurity level in mycophenolic acid fermentation was measured by HPLC and the final productivity was also measured. These results obtained from examples 1-6 are presented in Table 1. In examples 1, 2 and 3 mycophenolic acid fermentation was performed by standard fermentation procedure, i.e. batch fermentation where the level of the carbon source was not controlled. In example 4, mycophenolic acid fermentation was performed by fed-batch fermentation where the level of carbon source was controlled and feeding was stopped at the end of the fermentation. In examples 5 and 6, mycophenolic acid fermentation was performed by improved fed-batch fermentation where the level of carbon source was controlled and feeding was stopped at 24-48 hr before harvest.

TABLE 1
Comparison of results obtained from Examples 1 to 6

| | | | | |
|---|---|---|---|---|
| | | Intermediary | Final | Final |
| Example | | impurity level | impurity | productivity |
| No. | Fermentation | (120-230 h) [%] | level [%] | [g/l] |

Penicillium CCM 8364

| | | | | |
|---|---|---|---|---|
| 1 | Batch | 1-13 | 0.5 | 7.5 |
| 2 | Batch | 0-4.5 | 2-4 | 1.3 |
| 4 | Fed-batch | 1-4 | 0.5 | 9.9 |
| 5 | Improved | 1-4 | <0.15 | 10 |
| | fed-batch | | | |

P. brevicompactum ATCC16024

| | | | | |
|---|---|---|---|---|
| 3 | Batch | 1.4-3 | 0.9-1.2 | 1.8 |
| 6 | Improved | 0.1-1 | 0.2-0.3 | 2.2 |
| | fed-batch | | | |

The results in Table 1 demonstrate that the impurity level in mycophenolic acid was reduced considerably during fermentation of two strains, Penicillium CCM 8364 and P. brevicompactum ATCC16024, when it was carried out by fed batch fermentation and improved fed batch fermentation, where the level of carbon source was controlled, (examples 4-6), in comparison with using the standard batch fermentation procedure, where the level of carbon source was not controlled (examples 1-3). In addition, the final productivity and production rate of mycophenolic acid was increased when mycophenolic acid fermentation was carried by fed batch fermentation and improved fed batch fermentation (examples 4-6) in comparison with using the standard fermentation procedure (examples 1-3).

### Example 8

### Mycophenolate Mofetil Production from Mycophenolic Acid, According to International Patent Application Publication No. WO 2005/105771.

A mixture of mycophenolic acid (192 g, 0.6 mol) and 4-(2-hydroxyethyl)-morpholine (440 ml, 6 molar equivalents) is stirred at 150-155° C. for 4 hours in the presence of tin(II) chloride dihydrate (20.4 g, 0.15 molar equivalents) under nitrogen atmosphere. After the completion of the reaction, the reaction mixture is allowed to cool to room temperature. The obtained dark liquid is poured into isobutyl acetate (4.0 1). The solution is extracted with 2% of aqueous sodium bicarbonate solution (1.2 1, then 2×0.4 1). After the first addition of sodium bicarbonate solution the formed two-phase system is treated with charcoal (40 g) and filtrated (an emulsion was filtered off). The solution is extracted with water (1 1). After phase separation the organic phase is washed with water (1 1) and evaporated to dryness at 40-50° C. under vacuum. To the solid material acetone (400 ml) and isopropanol (3.8 1) are added and the mixture is warmed to 40-45 ° C. (the material is dissolved). The solution is cooled to −5° C. during 6 hours and it is stirred at this temperature for 10-12 hours. After filtration, the crystals are washed with 2:19 acetone/isopropanol mixture (420 ml). The crude compound is dried in vacuum at 60° C. The yield was 169-195 g (65-75%). HPLC impurity profile: MPA=0.1%. Assay: 99.85%.

### Example.9

### Preparation of Concentrated Mycophenolic Acid, According to Published US Patent Application No. 20050250952

Fermented broth (220 kg) is adjusted to approx. pH 8.0. The fermented broth is filtered by microfiltration plastic membranes (e.g. MFK-617 and HFM-180, by KOCH). Water is added continuously for dilution during filtration. The filtered broth is adjusted to approx. pH 4.0, and the crystal suspension is concentrated to approx. 70 liters. The pH of the concentrated acidic suspension is then adjusted to an alkaline pH of 7.5-11.0.

This alkaline suspension is used for purification of mycophenolic acid, such as in the following example.

### Example 10

### Purification of Mycophenolic Acid According to Published US Patent Application No. 20050250952:

A concentrated mycophenolic acid suspension of 140 kg (produced from 620 kg fermented broth) is adjusted with 800 ml conc. ammonium hydroxide solution to a pH of 8.3-8.5. The alkaline solution is purified with 80 liters of ethylacetate. The ethylacetate is mixed to the alkaline solution, stirred for 30 minutes, and the phases are separated.

To the obtained (147 kg) aqueous phase, 80 liters of ethylacetate are added. The pH is adjusted to 5.8 with sulfuric acid, stirred for 30 minutes, and the phases are separated.

To the obtained (150 kg) aqueous phase, 40 liters of ethylacetate are added. The pH is adjusted to 5.9, stirred for 30 minutes, and phases are separated.

The ethylacetate phases of the two acidic extractions are combined and concentrated to approx. 200 g/l concentration at max. 70° C. under reduced pressure. Concentrated ethylacetate solution is heated to 60-65° C., cooled to −10° C. at a cooling rate of approx. 3° C./hour, and allowed to crystallize for 18 hours at −10° C. The crystals are filtered and coverwashed with cooled ethylacetate. The crystals are dried at max. 70° C. under reduced pressure. Mass of crystals: 1250 g. Assay: 99.0%.

The crystals are recrystallized from ethylacetate after treatment with charcoal.

### Example 11

### Isolation of Homo-Mycophenolic Acid

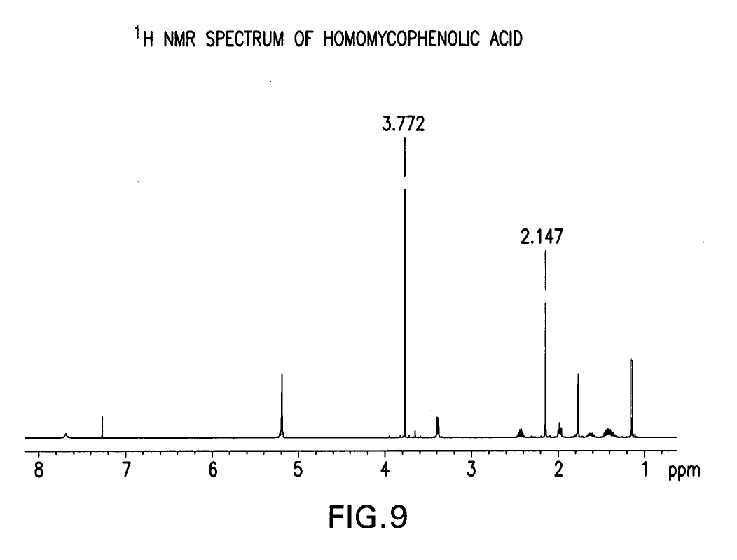

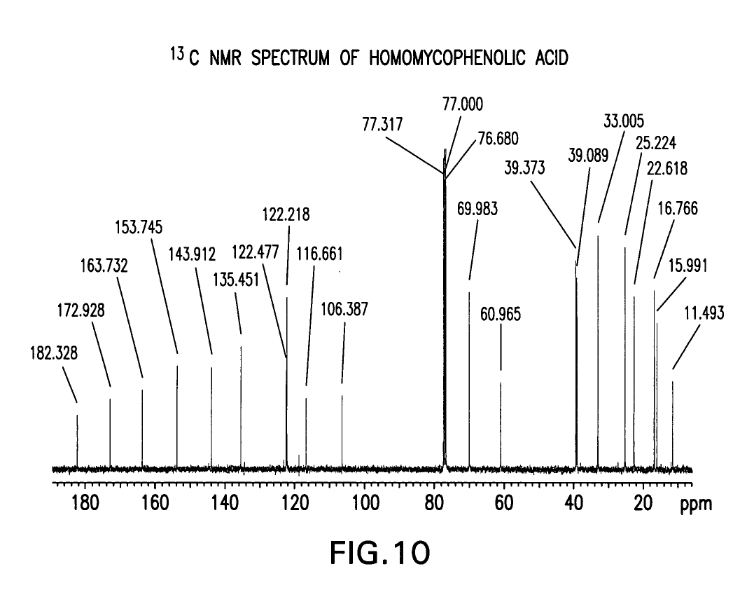

Concentrates from the production of mycophenolic acid (10 g), were purified by column chromatography on a silica gel using dichlormethane stepwise polarized with methanol (0-5% v/v). Fractions were monitored by TLC on a silica gel (benzene:acetic acid, 9:1 v/v). Fractions containing homomycophenolic acid (at 1.5% of methanol) were pooled and evaporated to dryness (3 g). Finally, the purification of homo-mycophenolic acid was carried out by preparative RP-HPLC (C-18 column and isocratic elution with 76% aqueous methanol, v/v, 9 ml/min). Purified homo-mycophenolic acid was eluted at 36.6-39.5 min. White crystalline homo-mycophenolic acid was obtained by crystallization from methanol/water (see FIGS. 9 and 10).

### Example 12

### Purification of Mycophenolic Acid

To 14 m³ of harvested fermented broth, the same volume of drinking water was added, followed by 168 liters (1.2%) of conc. ammonia solution. Filter aid (perlite) in 1% mass of the starting fermented broth was added, and the pH was adjusted to between 8.0-8.5 by adding conc. 85% phosphoric acid solution (approx. 100 liters). The treated broth was kept at ambient temperature without stirring for at least 6 hours. Filtration was carried out on vacuum drum filter during cover washing with drinking water. Filtrate of 42 m³ was collected. Yield from filtration of the fermented broth was approx. 90%.

The pH of the filtered fermented broth was adjusted to 4.0-4.5 by adding 20% sulfuric acid solution (approx. 300 liters). After at least 3 hours, the precipitated crude crystals were filtered and concentrated on microfiltration membrane (MFK-617, by KOCH). The pH-adjusted 42 m³ filtered fermented broth was concentrated to 1/40 volume (approx. 1.0-1.2 m³). The filtration time was approx. 60 hours. The concentrated solution was diluted with approx. 2 m³ acidic water, and the solution was concentrated again to 1.0-1.2 m³. After removing the concentrated solution, the equipment was washed with 0.3-0.5 m³ of acidic drinking water. Yield from precipitation and concentration was approx. 80%.

The 1.0-1.2 m³ concentrate and the 0.3-0.5 m³ acidic washing water were combined, 0.5-0.6 folds volume of ethylacetate is added (approx. 0.8 m³), and the pH was adjusted to between 9.0-9.2 with conc. ammonia solution. Extraction was carried out for 30 minutes, and the pH was adjusted to between 9.0-9.2. The phases were then separated.

To the aqueous phase, 0.5-0.6 folds volume of ethylacetate was added (approx. 0.8 m³) again (calculated to the volume of the combined acidic concentrate), and the pH was adjusted to between 9.0-9.2 with conc. ammonia solution/20% sulfuric acid solution. Extraction was carried out for 30 minutes, and the pH was adjusted to between 9.0-9.2. The phases were then separated.

To the aqueous phase, 0.5-0.6 folds volume of ethylacetate was added (calculated to the volume of the combined acidic concentrate), and the pH was adjusted to 5.8-6.1 with 20% sulfuric acid solution. Extraction was carried out for 30 minutes, and the pH was adjusted to between 5.8-6.1. The phases were then separated.

To the aqueous phase, 0.25-0.3 folds volume of ethylacetate was added (calculated to the volume of the combined acidic concentrate), and the pH was adjusted to 6.3-6.5 with conc. ammonia solution. Extraction was carried out for 30 minutes, and the pH was adjusted to between 6.3-6.5. The phases were then separated.

The third and fourth ethylacetate phases were combined and evaporated to approx. 200 g/l (based on evaporation residue of the combined phases) at max 70° C. under reduced pressure. The final volume of the evaporation was approx. 150 liters. Yield from extraction and evaporation was approx. 90%.

The ethylacetate concentrate (approx. 150 liters) was cooled to −10° C. to −17° C. (cooling rate approx. 3° C./hour), and crystallized at this temperature for at least 2 hours. The crystals were washed with 45 liters of chilled ethylacetate and dried at max. 70° C. under reduced pressure. Mass of the crystals was approx. 25 kg. Yield from crystallization was approx. 87%.

What is claimed is:

1. A process for reducing impurity formation during fermentation of mycophenolic acid (MPA) comprising: controlling the level of the carbon source during fermentation of MPA, wherein the carbon source is maintained at an amount of about 0.02% to about 0.8% w/w.

2. The process of claim 1, wherein the level of carbon source is maintained at an amount of about 0.05% to about 0.5% w/w.

3. The process of claim 1, wherein the impurities are selected from the group consisting of: MPA-IV of the following formula: 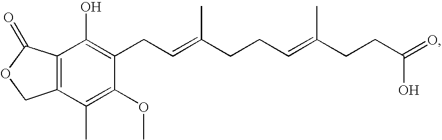
homo-MPA of the following formula: 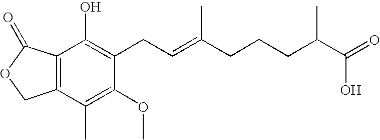
and combinations thereof.

4. The process of claim 1, wherein the level of the carbon source is maintained during production phase of the fermentation process.

5. The process of claim 1, wherein the carbon source is a carbohydrate.

6. The process of claim 5, wherein the carbon source is starch or molasses.

7. The process of claim 5, wherein the carbon source is at least one of glucose, sucrose, maltose or glycerol.

8. The process of claim 7, wherein the carbon source is glucose.

9. The process of claim 3, wherein the amount of MPA-IV in the obtained fermentation broth is less than about 0.5% area by HPLC.

10. The process of claim 3, wherein the amount of homo-MPA in the obtained fermentation broth is less than about 0.5% area by HPLC.

11. The process of claim 1, further comprising stop controlling the carbon source prior to harvesting.

12. The process of claim 11, wherein controlling the carbon source is stopped by stop feeding the carbon source during final 20 to 96 hours of production stage.

13. The process of claim 1, comprising a fermentation medium containing a starting level of a carbon source (growth period) which is about 1% to about 5% w/w of the fermentation medium.

14. The process of claim 1, comprising a fermentation broth wherein at least one of, a mineral salt, a source of nitrogen, a microbial growth factor, a source of phosphorous, or a buffer is added to the fermentation broth.

15. The process of claim 14, wherein the mineral salt is at least one of magnesium sulfate, manganese dichloride, ferrous sulfate, zinc chloride, copper II sulfate, ammonium sulfate, potassium dihydrogen phosphate, sodium chloride or calcium carbonate.

16. The process of claim 14, wherein the nitrogen source is an assimilable organic or inorganic nitrogen source.

17. The process of claim 14, wherein the nitrogen source is a nitrate, urea, an ammonium salt, corn steep liquor or an amino acid.

18. The process of claim 15, wherein the microbial growth factor is at least one of yeast extract, or vitamins.

19. The process of claim 15, wherein the phosphorous source is potassium dihydrogen phosphate.

20. The process of claim 1, wherein fermentation of mycophenolic acid is by a mycophenolic acid producing micro-organism selected from the group consisting of P. brevi-compactum, P. scabrum, P. nagemi, P. roqueforti, P. patris-mei and P. viridicatum or a derivative thereof.

21. The process of claim 20, wherein the mycophenolic acid producing micro-organism is the Penicillium sp. strain (accession number CCM 8364) or a derivative thereof.

22. The process of claim 1, further comprising isolating MPA from the fermentation broth.

23. The process of claim 1, further comprising batchwise feeding of the nitrogen source.

24. The process of claim 23, wherein the nitrogen source is glycine and/or corn steep liquor to the fermentation broth.

25. A process for preparing MMF having a reduced level of impurities comprising preparing MPA according to the process of claim 1 and converting it to MMF.

26. Isolated E-8-(4-hydroxy-6-methoxy-7-methyl-3-oxo-1,3-dihydro-isobenzofuran-5-yl)-2,6-dimethyl-oct-6-enoic acid (“homo-MPA”) of the following formula:

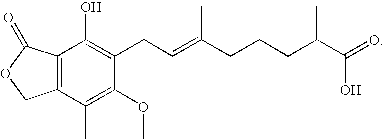

27. The isolated Homo-MPA of claim 26, wherein it is a solid.

28. The isolated Homo-MPA of claim 27, wherein it is crystalline.

29. The isolated homo-mycophenolic acid of claim 26 characterized by data selected from a group consisting of: ¹H NMR (400 MHz, CDCl₃) δ (ppm): 1.15, 1.35, 1.42, 1.63, 1.77, 1.98, 2.15, 2.43,3.39, 3.77, 5.19, 5.20, and 7.68; ¹³C NMR (100 MHz, CDCl₃) δ (ppm): 11.5,16.0, 16.8, 22.6, 25.2,33.0,39.1, 39.4, 61.0, 70.0106.4, 116.7, 122.2, 122.5, 135.5143.9, 153.7, 163.7, 172.9, and 182.3, and combination thereof.

30. The isolated homo-mycophenolic acid of claim 26 characterized by data selected from a group consisting of: an ¹H NMR spectrum as depicted in FIG. 6, a ¹³C NMR spectrum as depicted in FIG. 7 and combination thereof.

31. The isolated homo-MPA of claim 26, wherein it is a homo-MPA composition comprising MPA in an amount of less than about 5% by weight.

32. The composition of claim 31, wherein the homo-MPA composition contains about 5% to about 0.05% by weight of MPA.

33. A process of determining the presence of homo-MPA in a sample of MPA comprising carrying out HPLC or TLC of a sample of MPA with the homo-MPA as a reference marker.

34. The process of claim 33 comprising (a) measuring by HPLC or TLC the relative retention time or factor corresponding to the homo-MPA in a reference marker sample; (b) determining by HPLC or TLC the relative retention time corresponding to homo-MPA in a sample comprising homo-MPA and MPA; and (c) identifying homo-MPA in the sample by comparing the relative retention time or factor of homo-MPA as measured in step (a) to the RRT or RRF of step (b).

35. A process of determining the amount of homo-MPA in a sample of MPA comprising homo-MPA and MPA by a process comprising carrying out HPLC with homo-MPA as a reference standard.

36. The process of claim 35 comprising (a) measuring by HPLC the area under a peak corresponding to the homo-MPA in a reference standard comprising a known amount of homo-MPA; (b) measuring by HPLC the area under a peak corresponding to homo-MPA in a sample comprising homo-MPA and MPA; and (c) determining the amount of homo-MPA in the sample by comparing the area of step (a) to the area of step (b).

37. A method for preparing mycophenolic acid comprising: preparing a fermentation broth containing mycophenolic acid producing micro-organism; fermenting of mycophenolic acid while feeding nutrients to the fermentation broth; controlling the level of carbon source in the fermentation broth at an amount of about 0.02% to about 0.8% w/w during the fermentation stage of mycophenolic acid; harvesting and recovering the mycophenolic acid from the fermentation broth.

38. An isolated compound of following structure: [chemical structure]
